(19) United States
(12) Patent Application Publication
(10) Pub. No.: US 2023/0326583 A1
(43) Pub. Date: Oct. 12, 2023

(54) PERSONALIZED WORKLIST SORTING SYSTEM FOR MEDICAL IMAGE VIEWING BY MEDICAL PROFESSIONALS

(71) Applicant: INTERNATIONAL BUSINESS MACHINES CORPORATION, ARMONK, NY (US)

(72) Inventors: Sun Young Park, San Diego, CA (US); Dustin Michael Sargent, San Diego, CA (US); William Kazee, Brooksville, FL (US); TROY OLIPHANT, Kansas City, MO (US); Giovanni John Jacques Palma, Chaville (FR)

(21) Appl. No.: 17/656,440

(22) Filed: Mar. 25, 2022

(51) Int. Cl. G16H 40/20; G06Q 10/10; G16H 10/60

(52) U.S. Cl. CPC G16H 40/20; G06Q 10/1097; G16H 10/60

(57) ABSTRACT

Computer technology for scheduling viewing of sets of medical images for evaluation of medical images (for example X-ray images) by a medical professional (for example, a radiologist). The scheduling is based on, at least in part, scheduling rules obtained from computerized analysis of historical viewing patterns of the medical professional and/or other similarly situated medical professional viewers. The scheduling may include various types of scheduling, such as time of day scheduling, date scheduling (that is, day of the week / month / year scheduling, order of images to be viewed within a set of patient images, order of viewing among and between the various patient sets of images and amount of time to be spent on each image, each set of images.

### BACKGROUND

The present invention relates to radiology and also to picture archiving and communications systems (PACS) used by radiologists, and similar medical professionals to view medical images.

The Wikipedia entry for “Radiology” (as of Jan. 31, 2020) states, in part, as follows: “Radiology is the medical discipline that uses medical imaging to diagnose and treat diseases within the bodies of animals and humans. A variety of imaging techniques such as X-ray radiography, ultrasound, computed tomography (CT), nuclear medicine including positron emission tomography (PET), fluoroscopy, and magnetic resonance imaging (MRI) are used to diagnose or treat diseases. Interventional radiology is the performance of usually minimally invasive medical procedures with the guidance of imaging technologies such as those mentioned above ... The modern practice of radiology involves several different healthcare professions working as a team. The radiologist is a medical doctor who has completed the appropriate postgraduate training and interprets medical images, communicates these findings to other physicians by means of a report or verbally, and uses imaging to perform minimally invasive medical procedures... The radiographer... is a specially trained healthcare professional that uses sophisticated technology and positioning techniques to produce medical images for the radiologist to interpret. Depending on the individual’s training and country of practice, the radiographer may specialize in one of the above-mentioned imaging modalities or have expanded roles in image reporting.” (footnote(s) omitted)

The Wikipedia entry for “Picture archiving and communication system” (as of Jan. 31, 2022) states, in part, as follows: “A picture archiving and communication system (PACS) is a medical imaging technology which provides economical storage and convenient access to images from multiple modalities (source machine types). Electronic images and reports are transmitted digitally via PACS; this eliminates the need to manually file, retrieve, or transport film jackets, the folders used to store and protect X-ray film. The universal format for PACS image storage and transfer is DICOM (Digital Imaging and Communications in Medicine). Non-image data, such as scanned documents, may be incorporated using consumer industry standard formats like PDF (Portable Document Format), once encapsulated in DICOM. A PACS consists of four major components: The imaging modalities such as X-ray plain film (PF), computed tomography (CT) and magnetic resonance imaging (MRI), a secured network for the transmission of patient information, workstations for interpreting and reviewing images, and archives for the storage and retrieval of images and reports. Combined with available and emerging web technology, PACS has the ability to deliver timely and efficient access to images, interpretations, and related data. PACS reduces the physical and time barriers associated with traditional film-based image retrieval, distribution, and display.” (footnote(s) omitted)

Many types of medical images are now known, and it is highly likely that even more types of medical images will be developed in the future. For purposes of this document, a medical image is any graphic images corresponding to a patient’s medical, bodily and/or physiological related data. For example, an x-ray is a medical image that is essentially a picture of a portion of the patient’s body. Other medical images, like various medical graphs, correspond to numbers, such as heart function metrics. Some currently known types of medical images include: CR - Computer Radiography, CT - Computed Tomography, MR - Magnetic Resonance, NM - Nuclear Medicine, US - Ultrasound, PT - Positron Emission Tomography, XA - X-Ray Angiography, DX - Digital Radiography, MG - Mammography, ECG -Electrocardiography and HD - Hemodynamic Waveform.

“Medical image sets” (or “patient image sets”), as that term is used herein, refers to a set of medical images that a medical professional would look at the same time, typically according to customs and practices in their specific field of medicine or medical research. “Studies” are a kind of patient image set that have images from several different individuals. For other medical professionals, their image sets may be typically allocated for review an/or for scheduling purposes, so that each individual image is viewed in an independent fashion -meaning that each image set would have only a single image for purposes of scheduling that sort of reviewer. Conventionally, “studies” are a kind of patient image set that have images from several different individuals, and “exams” are patient image sets with images related to a single individual. For purposes of this document, it does not matter whether the patient image sets to be reviewed are in the forms of studies, exams or a mixture of these two types.

### SUMMARY

According to an aspect of the present invention, there is a method, computer program product and/or system for use by a first medical profession who reviews patient image sets, respectively including at least one medical image, for medical purposes that performs the following operations (not necessarily in the following order): (i) receiving a historical image review data set including information indicative of: (a) the manner in which the first medical professional has scheduled review of patient data image sets, and (b) the manner in which a plurality of medical professionals have scheduled review of patient image sets; (ii) determining, by machine logic, a set of at least one personalized scheduling rule(s) based on the historical image data review set; (iii) receiving a first plurality of patient image sets respectively including one or medical image(s) of a respective patient; and (iv) scheduling review of the first plurality of patient image sets based, at least in part, upon the personalized scheduling rules to obtain a first schedule for a first time period for the first medical professional to review the first plurality of patient image sets.

According to an aspect of the present invention, there is a method, computer program product and/or system for use by a first medical profession who reviews patient image sets, respectively including at least one medical image, for medical purposes that performs the following operations (not necessarily in the following order): (i) receiving a historical image review data set including information indicative of the manner in which the first medical professional has scheduled review of patient data image sets; (ii) determining, by machine logic, a set of at least one personalized scheduling rule(s) based on the historical image data review set; (iii) receiving a first plurality of patient image sets respectively including one or medical image(s) of a respective patient; and (iv) scheduling review of the first plurality of patient image sets based, at least in part, upon the personalized scheduling rules to obtain a first schedule for a first time period for the first medical professional to review the first plurality of patient image sets.

According to an aspect of the present invention, there is a method, computer program product and/or system for use by a first medical profession who reviews patient image sets, respectively including at least one medical image, for medical purposes that performs the following operations (not necessarily in the following order): (i) receiving a historical image review data set including information indicative of the manner in which a plurality of medical professionals have scheduled review of patient image sets; (ii) determining, by machine logic, a set of at least one personalized scheduling rule(s) based on the historical image data review set; (iii) receiving a first plurality of patient image sets respectively including one or medical image(s) of a respective patient; and (iv) scheduling review of the first plurality of patient image sets based, at least in part, upon the personalized scheduling rules to obtain a first schedule for a first time period for the first medical professional to review the first plurality of patient image sets.

### BRIEF DESCRIPTION OF THE DRAWINGS

FIG. 1 is a block diagram view of a first embodiment of a system according to the present invention;

FIG. 2 is a flowchart showing a first embodiment method performed, at least in part, by the first embodiment system;

FIG. 3 is a block diagram showing a machine logic (for example, software) portion of the first embodiment system;

FIG. 4 is a screenshot view generated by the first embodiment system;

FIG. 5 is a first diagram of a second embodiment of a system according to the present invention;

FIG. 6 is a diagram helpful in understanding various embodiments of the present invention;

FIG. 7 is another diagram helpful in understanding various embodiments of the present invention; and

FIG. 8 is another diagram helpful in understanding various embodiments of the present invention.

### DETAILED DESCRIPTION

Some embodiments are directed to computer technology for scheduling viewing of sets of medical images for evaluation of medical images (for example X-ray images) by a medical professional (for example, a radiologist). The scheduling is based on, at least in part, scheduling rules obtained from computerized analysis of historical viewing patterns of the medical professional and/or other similarly situated medical professional viewers. The scheduling may include various types of scheduling, such as time of day scheduling, date scheduling (that is, day of the week / month / year scheduling, order of images to be viewed within a set of patient images, order of viewing among and between the various patient sets of images and amount of time to be spent on each image, each set of images. This Detailed Description section is divided into the following subsections: (i) The Hardware and Software Environment; (ii) Example Embodiment; (iii) Further Comments and/or Embodiments; and (iv) Definitions.

### I. The Hardware and Software Environment

The present invention may be a system, a method, and/or a computer program product at any possible technical detail level of integration. The computer program product may include a computer readable storage medium (or media) having computer readable program instructions thereon for causing a processor to carry out aspects of the present invention. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire.

A “storage device” is hereby defined to be anything made or adapted to store computer code in a manner so that the computer code can be accessed by a computer processor. A storage device typically includes a storage medium, which is the material in, or on, which the data of the computer code is stored. A single “storage device” may have: (i) multiple discrete portions that are spaced apart, or distributed (for example, a set of six solid state storage devices respectively located in six laptop computers that collectively store a single computer program); and/or (ii) may use multiple storage media (for example, a set of computer code that is partially stored in as magnetic domains in a computer’s non-volatile storage and partially stored in a set of semiconductor switches in the computer’s volatile memory). The term “storage medium” should be construed to cover situations where multiple different types of storage media are used.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

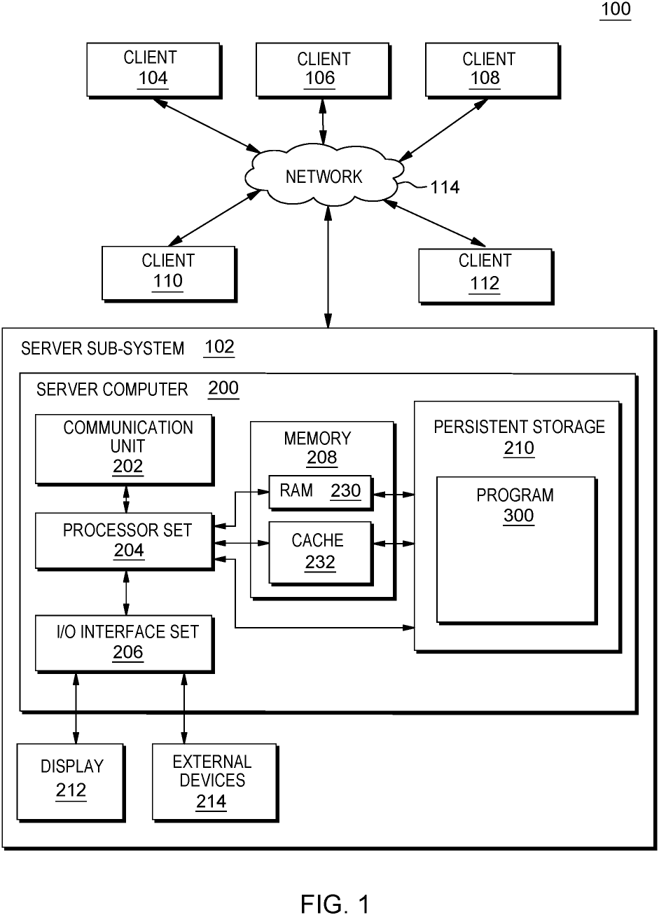

As shown in FIG. 1, networked computers system 100 is an embodiment of a hardware and software environment for use with various embodiments of the present invention. Networked computers system 100 includes: server subsystem 102 (sometimes herein referred to, more simply, as subsystem 102); client subsystems 104, 106, 108, 110, 112; and communication network 114. Server subsystem 102 includes: server computer 200; communication unit 202; processor set 204; input/output (I/O) interface set 206; memory 208; persistent storage 210; display 212; external device(s) 214; random access memory (RAM) 230; cache 232; and program 300.

Subsystem 102 may be a laptop computer, tablet computer, netbook computer, personal computer (PC), a desktop computer, a personal digital assistant (PDA), a smart phone, or any other type of computer (see definition of “computer” in Definitions section, below). Program 300 is a collection of machine readable instructions and/or data that is used to create, manage and control certain software functions that will be discussed in detail, below, in the Example Embodiment subsection of this Detailed Description section.

Subsystem 102 is capable of communicating with other computer subsystems via communication network 114. Network 114 can be, for example, a local area network (LAN), a wide area network (WAN) such as the Internet, or a combination of the two, and can include wired, wireless, or fiber optic connections. In general, network 114 can be any combination of connections and protocols that will support communications between server and client subsystems.

Subsystem 102 is shown as a block diagram with many double arrows. These double arrows (no separate reference numerals) represent a communications fabric, which provides communications between various components of subsystem 102. This communications fabric can be implemented with any architecture designed for passing data and/or control information between processors (such as microprocessors, communications and network processors, etc.), system memory, peripheral devices, and any other hardware components within a computer system. For example, the communications fabric can be implemented, at least in part, with one or more buses.

Memory 208 and persistent storage 210 are computer-readable storage media. In general, memory 208 can include any suitable volatile or non-volatile computer-readable storage media. It is further noted that, now and/or in the near future: (i) external device(s) 214 may be able to supply, some or all, memory for subsystem 102; and/or (ii) devices external to subsystem 102 may be able to provide memory for subsystem 102. Both memory 208 and persistent storage 210: (i) store data in a manner that is less transient than a signal in transit; and (ii) store data on a tangible medium (such as magnetic or optical domains). In this embodiment, memory 208 is volatile storage, while persistent storage 210 provides nonvolatile storage. The media used by persistent storage 210 may also be removable. For example, a removable hard drive may be used for persistent storage 210. Other examples include optical and magnetic disks, thumb drives, and smart cards that are inserted into a drive for transfer onto another computer-readable storage medium that is also part of persistent storage 210.

Communications unit 202 provides for communications with other data processing systems or devices external to subsystem 102. In these examples, communications unit 202 includes one or more network interface cards. Communications unit 202 may provide communications through the use of either or both physical and wireless communications links. Any software modules discussed herein may be downloaded to a persistent storage device (such as persistent storage 210) through a communications unit (such as communications unit 202).

I/O interface set 206 allows for input and output of data with other devices that may be connected locally in data communication with server computer 200. For example, I/O interface set 206 provides a connection to external device set 214. External device set 214 will typically include devices such as a keyboard, keypad, a touch screen, and/or some other suitable input device. External device set 214 can also include portable computer-readable storage media such as, for example, thumb drives, portable optical or magnetic disks, and memory cards. Software and data used to practice embodiments of the present invention, for example, program 300, can be stored on such portable computer-readable storage media. I/O interface set 206 also connects in data communication with display 212. Display 212 is a display device that provides a mechanism to display data to a user and may be, for example, a computer monitor or a smart phone display screen.

In this embodiment, program 300 is stored in persistent storage 210 for access and/or execution by one or more computer processors of processor set 204, usually through one or more memories of memory 208. It will be understood by those of skill in the art that program 300 may be stored in a more highly distributed manner during its run time and/or when it is not running. Program 300 may include both machine readable and performable instructions and/or substantive data (that is, the type of data stored in a database). In this particular embodiment, persistent storage 210 includes a magnetic hard disk drive. To name some possible variations, persistent storage 210 may include a solid state hard drive, a semiconductor storage device, read-only memory (ROM), erasable programmable read-only memory (EPROM), flash memory, or any other computer-readable storage media that is capable of storing program instructions or digital information.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### II. Example Embodiment

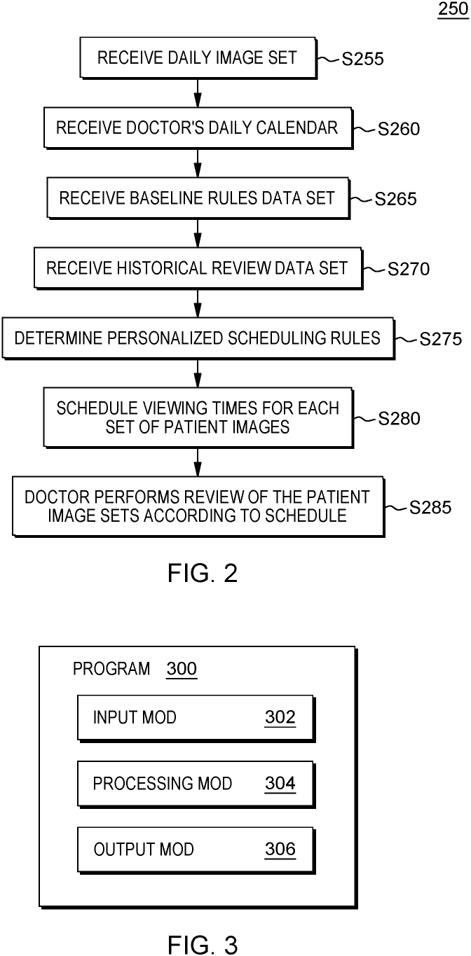

As shown in FIG. 1, networked computers system 100 is an environment in which an example method according to the present invention can be performed. As shown in FIG. 2, flowchart 250 shows an example method according to the present invention. As shown in FIG. 3, program 300 performs or controls performance of at least some of the method operations of flowchart 250. This method and associated software will now be discussed, over the course of the following paragraphs, with extensive reference to the blocks of FIGS. 1, 2 and 3.

Processing begins at operation S255, where input module (“mod”) 302 receives a daily image set that includes all of the X-ray images that a radiologist is going to look at on the date 02 Jan. 2024. More specifically, the daily image data set includes images as described in the following table:

| PATIENT | URGENCY | No. IMAGES | ALLOTTED TIME | AGE |
|---|---|---|---|---|
| A | 2 | 6 | 60 MINUTES | 11 |
| B | 1 | 15 | 60 MINUTES | 88 |
| C | 2 | 14 | 30 MINUTES | 5 |
| D | 3 | 2 | 15 MINUTES | 2 |
| E | 3 | 1 | 30 MINUTES | 8 |
| F | 2 | 25 | 60 MINUTES | 32 |
| G | 2 | 1 | 15 MINUTES | 3 |
| H | 3 | 3 | 15 MINUTES | 55 |
| I | 2 | 66 | 90 MINUTES | 44 |
| J | 3 | 66 | 90 MINUTES | 91 |
| K | 2 | 8 | 30 MINUTES | 78 |
| L | 2 | 75 | 120 MINUTES | 50 |

It is noted that real world radiologists would never encounter such voluminous image sets as set forth in the table above, and/or would never encounter such a spread in image set sizes as set forth in the table above. This example is presented for pedagogical purposes and is not necessarily intended to be particularly realistic with respect to any particular type of medicalpractitioner. For example, in some embodiments, a set of exams (exams rather than images, as an exam may contain multiple images) would typically not include the entire set of exams for the whole day. In some embodiments, exams come in gradually throughout the day. In some embodiments, a whole day’s exam list would not be available before the workday begins (as it is in the embodiment of flowchart 250). In some embodiments, it is understood that a given radiologist’s preferences may vary throughout the day, so that is taken into into account based on when the scheduling request arrives. Some embodiments do not explicitly dictate what time a radiologist to has read something and how long it should take.

Some embodiments may schedule time slots more loosely, so that the “allotted time” is taken to be an estimated reading time that can vary on a case by case basis. An example of an estimated reading time range might be 1 - 10 minutes. Some embodiments do not even include the an estimated or an allotted time in the scheduling, but, rather, perform a more limited form of scheduling where the schedule only dictates the optimal ordering of the image sets to be reviewed by the medical practitioner over the time period covered by the schedule.

In the example, above, the patient attribute that is available to base rules upon is age. As discussed elsewhere herein, other types of patient attributes and/or other attributes relating to other aspects of the image review may be used, such as an imaging modality attribute and/or an RVU attribute. While the pedagogical embodiment of flow chart 250 uses a single imaging modality (X-rays) to keep things simple, many embodiments will include additional modalities, such as MR and CT.

In this embodiment, all of the medical images are X-ray images and there is a single radiologist reviewing all of the images on a single workday. Alternatively: (i) the images may be picture based (like an x-ray), or they may take more abstract graphical forms, such as various types of graphs or charts; (ii) there may be more than one type of medical image; (iii) the scheduling period may be for a period longer or shorter than a day (for example, scheduling for a week long period); (iv) the scheduling may be made for a team of medical professionals working in co-operation instead of scheduling for a single radiologist; and/or (iv) the information about each set of patient X-rays may include different attributes about the X-ray set than what is included in the table above (for example, a precalculated amount of time to review each patient set might be omitted so that amount of time is calculated at the same time the rest of the scheduling operations (see below) are performed).

It is further noted that this embodiment is based on the following assumptions about this hypothetical radiologist’s work situation: (i) a set of images from a single patient will be looked at in a continuous stretch that is not interrupted by reviewing images of other patients; and (ii) the order that the radiologist reviews the images in a given patient set is not dictated by the software (that is, the radiologist can flip back and forth at will among and between the images of a given patient set. This idea of patient sets of images may be treated differently in some other embodiments for different medical review contexts. For example, in some embodiments, each individual image is treated as a patient set having a single image - either because each image does come from a different patient, or because there is no need to look at various different images from the same patient at the same time.

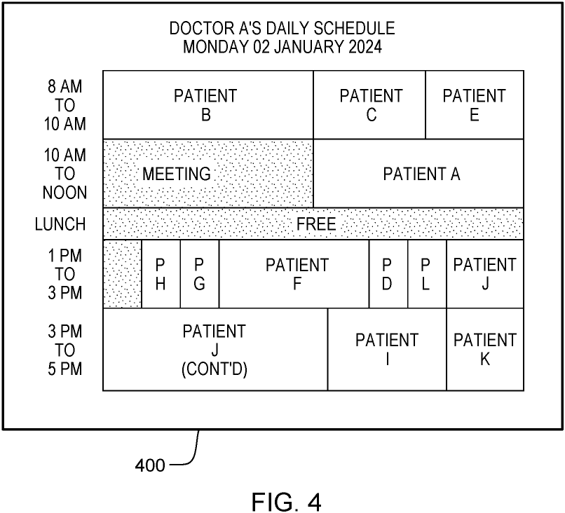

Processing proceeds to operation S260, where the input mod receives the doctor’s calendar data set. As shown in screenshot 400 of FIG. 4, this calendar set is for a single workday, and it is pre-populated with a meeting (see 10 am to 11 am slot in screen shot 400) and a meal break from NOON to 1 PM. It should be understood that not all embodiments will necessarily get / utilize calendar data. Also, in some embodiments, break periods may be learned over time from historical behavior patterns of a given radiologist or set of radiologists.

Processing proceeds to operation S265, where the input mod receives a baseline rules data set. These are scheduling rules that are to be applied prior to and/or in conjunction with the machine logic based (for example machine learning based) rules that will be determined later in the method of flow chart 250. In this example, the baseline rules are: (i) any reviews having an urgency level of “1” must be heard at the earliest possible opening, without consideration of other triage factors; and (ii) any unfilled time should be placed to the end of the lunch break, so long as that does not conflict with other scheduling rules (baseline scheduling rules, personalized scheduling rules)..

Processing proceeds to operation S270, where input mod 302 receives a historical review data set that includes historical data indicating how historical medical image review sessions have been scheduled / performed by medical professionals. More specifically, in this case, the historical review data set includes three types of historical data: (i) data on how the radiologist of this example has scheduled review of X-rays in the past (note: this kind of data is rather limited because it relates to the work of a single person); (ii) data on how other radiologists, and/or teams of radiologists, have scheduled review of X-rays in the past; and (iii) data on how other medical professionals, and/or teams of medical professionals, have scheduled review of magnetic resonance images (MRIs) in the past.

Processing proceeds to S275, where a machine learning algorithm of processing mod 304 determines personalized scheduling rules to be applied when scheduling review of the medical images identified in the daily image set. The determination of the personalized scheduling rules being based on the data of the historical review data set and machine learning (ML). In many embodiments, these rules take the form of patterns that may be difficult, or impossible, to express in a form that is readily understandable for human readers. Therefore, in this pedagogical and hypothetical example, the rules will be expressed as simple human understandable rules that have been drawn from patterns present in historical image reviews. In this example, three personalized rules are thus determined: PERSONALIZED RULE ONE: When feasible, image sets of patients under 12 years of age should be scheduled in the morning. PERSONALIZED RULE TWO: When feasible, image sets of patients over 70 years of age should be scheduled in the afternoon. PERSONALIZED RULE THREE: When feasible, fifteen (15) minute image sets should be scheduled in clusters of no less than two consecutive 15 minute image sets and no more than three consecutive 15 minute image sets. It is noted that the first two rules relate to scheduling within a day, relative to the time framework of a single workday for a single person. Alternatively, the scheduled period could be longer and/or include more than a single team member. The last of the personalized rules relates to how various patient image set reviews are scheduled relative to each other within a day.

In the foregoing example, the attributes of the patient image data sets used to make the triggering conditions for the personalized rules are: patient age and time allotment for review session of the patient image set. Additionally, or alternatively, many of other attributes could be used in defining triggering condition(s) for a personalized rule. These other attributes may include: (i) type of medical image (for example, X-rays versus MRI); (ii) average size of medical image in the image set; (iii) type of medical condition suspected (for example, a broken rib versus a broken finger); and/or (iv) average image resolution of the images in the image set.

This method of flowchart 250 determines a favorable (for example, optimal) ordering for sets of patient images already assigned to a specific radiologist. The personalized rules are personalized for a particular individual person, and not for a set of people working together in a team. Some personalized rules, according to various embodiments of the present invention may take into account things like: what does this particular user like to do in the morning versus the afternoon? When do they usually take their lunch break (to avoid scheduling large tasks right before that and avoid interruptions)? Does this user like to read many of the same exam type in a row or do they prefer variety? Some embodiments also consider how those factors affect the specific user’s diagnostic accuracy and efficiency. For example, some embodiments can detect that a radiologist’s diagnostic accuracy degrades over time when they read more than 5 CT images in a row.

Processing proceeds to operation S280, where processing mod 304 schedules review of the patient image sets for patients A to L set for thin the table above. It is noted that there was not sufficient time to allow patient L to be scheduled on 02 Jan. 2024, so that image set will be forwarded to another radiologist. The schedule thus determined is shown in screen shot 400 of FIG. 4. A comparison of the table of daily image sets for patients A to K with the schedule shows that the schedule is based in part on: (i) the doctor’s calendar data set (see, screenshot 400 at “MEETING” and “LUNCH”; (ii) the baseline rules (see, for example, the high priority accorded to PATIENT B); and (iii) the three (3) personalized rules determined by the machine learning algorithm based on the historical review data set. The schedule for the daily image data set of 02 Jan. 2024 is sent to the radiologist’s workspace in a hospital at client sub-system 104.

Processing proceeds to operation S285, using output mod 306, where the radiologist performs the review of the patient image sets for patients A to L on 02 Jan. 2024 according to the schedule shown in screen shot 400.

### III. Further Comments And/or Embodiments

Some embodiments of the present invention recognize the following facts, potential problems and/or potential areas for improvement with respect to the current state of the art: (i) radiologists read medical imaging studies from worklists; (ii) radiologists may have their own routines/preferences for reading order; (iii) accuracy, bias, and efficiency can be affected by worklist ordering; (iv) breaks, interruptions affect reading workflow; and/or (v) users can currently sort based on priority (due time), assigned time, or other study characteristics.

Some embodiments of the present invention recognize the following facts, potential problems and/or potential areas for improvement with respect to the current state of the art: (i) current PACS do not provide personalized worklist sorting functionality based on radiologists’ daily reading patterns; (ii) this adds manual sorting work or causes suboptimal, non-personalized scheduling and/or ordering reading of medical images by a medical professional; (iii) behavior changes are not reflected; (iv) efficient reading order is not analyzed; and/or (v) reading quality for the complex studies can be improved by fewer interruptions.

Some embodiments of the present invention may include one, or more, of the following operations, features, characteristics and/or advantages: (i) includes a personalized worklist sorting system that allows users to sort based on efficiency, accuracy, preference, or other factors, while also taking personal routines and sub-specialties into account; (ii) analyzes radiologist reading patterns; (iii) provides different sorting options including accuracy maximizing sort, fast reading sort, etc.; (iv) interactive adjustment and reorder; (v) personalized worklist sorting feature for PACS; (vi) interactive adjustment based on user’s routine behavior; and/or (vi) efficient reading order suggestion based on other radiologist’s reading patterns.

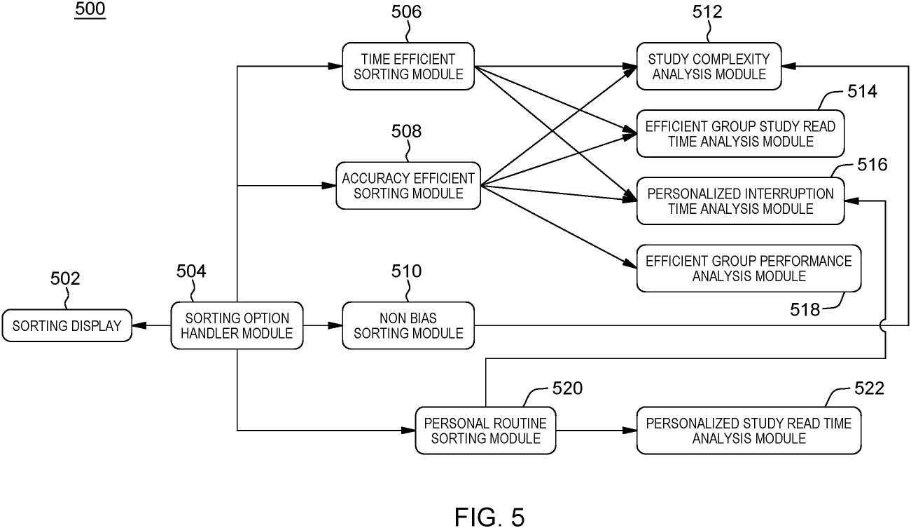

As shown in FIG. 5, system 500 includes: sorting display block 502; sorting option handler module 504; time efficient sorting module 506; accuracy efficient sorting module 508; non bias sorting module 510; study complexity analysis module 512; efficient group study read time analysis module 514; personalized interruption time analysis module 516; efficient group performance analysis module 518; personal routine sorting module 520; and personalized study read time analysis module 522.

A method for personalized ordering includes the following operations: (i) given a set of radiologists’ daily exam reading data, including (but not limited to): (a) modality, body part, RVU (relative value unit), study difficulty estimation, study description, other patient and study information (if available), and/or (b) orders exams to be read, interruptions and break times if available; (ii) trains a sequence model to learn the most preferred reading sequence; and/or (iii) optional: includes a post-processing output sequence based on a personal break schedule and likely interruption times including a way to: (a) detect if large blocks of work overlap break times, (b) adjust exam ordering to avoid breaks interrupting longer exam reading tasks, and/or (c) find block of tasks that fit available time slots and shifts to morning/afternoon so that the large task is uninterrupted.

A method of sorting with accuracy/efficiency includes the following operations: (i) given a set of radiologists’ daily exam reading data divided by sub-specialty, including (but not limited to): (a) modality, body part, RVU, study difficulty estimation, study description, other patient and study information (if available), (b) orders exams to be read and turnaround time for each exam, interruptions and break times, and/or (c) diagnostic accuracy information (pathology/biopsy results, peer review scores, follow-up results, etc.); (ii) from all sequences for a given sub-specialty, select the top X% for efficiency and accuracy; (iii) train sequence models for efficiency and accuracy as before on the selected top sequences from multiple radiologists; and/or (iv) optional: adjust output sequence for personal break times / interruptions for the target radiologist.

Training data: a set of daily exam reading sequences for each radiologist. Inference: given a radiologist and a set of exams, sort the exams into the optimal reading order for that radiologist. The following five paragraphs describe operations for a first method that uses a sequence model.

First operation: State: radiologist’s remaining capacity, last N exam types read (example N=3), and set of exams still to be placed in order.

Second operation: From training data, calculate probability of state transitions

Third operation: Build a directed acyclic graph (that is, using breadth first method) where the above states are nodes and edges are weighted by the state transition probabilities

Fourth operation: Optional: include break / interruption periods in the graph, or handle with post-processing

Fifth operation: To find the correct exam ordering: either use greedy method (fast, less accurate), or dynamic programming to find the global optimum path through the graph (slower)

The following three paragraphs describe operations for a second method that uses a sequence model.

First operation: States and transitions as above, but states contain only the previous exam instead of last N exams

Second operation: Build graph as above, but edge weights are adjusted by a cost for reading the same exam type twice in a row, cost personalized based on radiologist’s preference for doing so.

Third operation: Find optimal exam order (as described in operation 5 above).

The following four paragraphs describe operations for a third method that uses a sequence model.

First operation: Build probability distribution for radiologist’s next exam selection preference in each state using the training data.

Second operation: Optional: add time constraints based on break times / interruptions.

Third operation: Find optimal exam ordering subject to constraints by convolution of PDFs (portable document format).

Fourth operation: Can find maximal convolution sequence using dynamic programming.

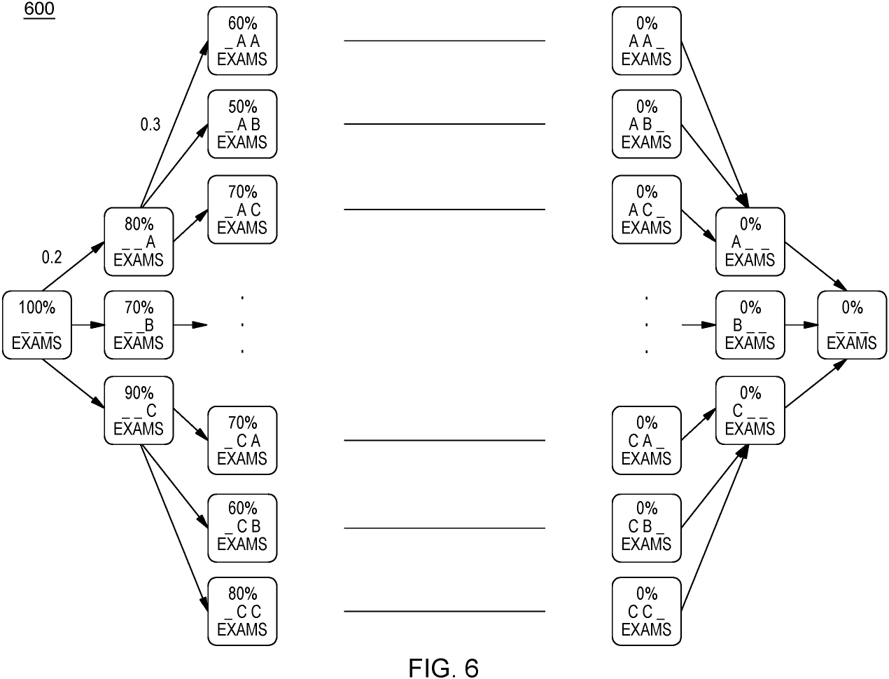

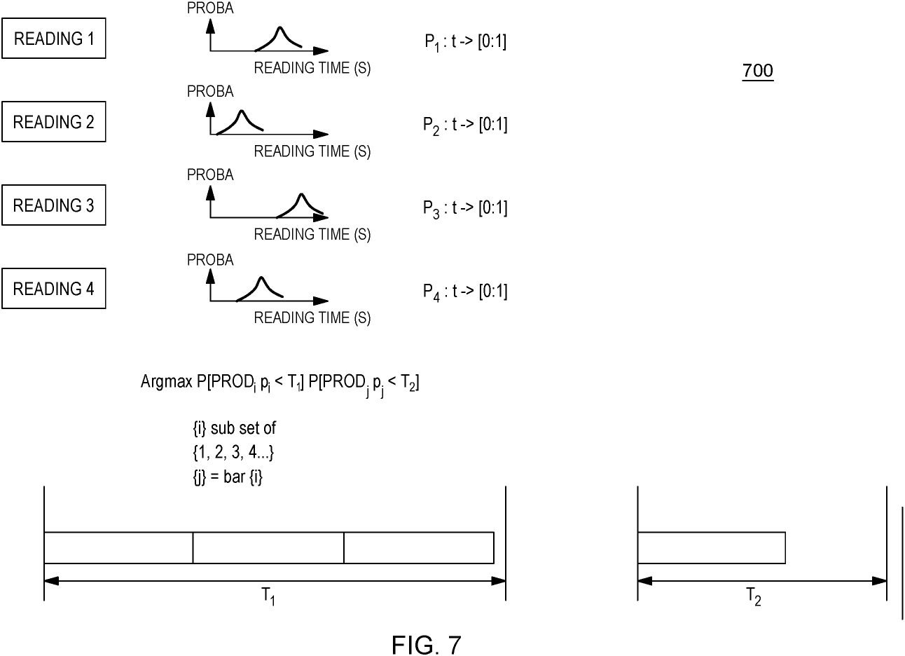

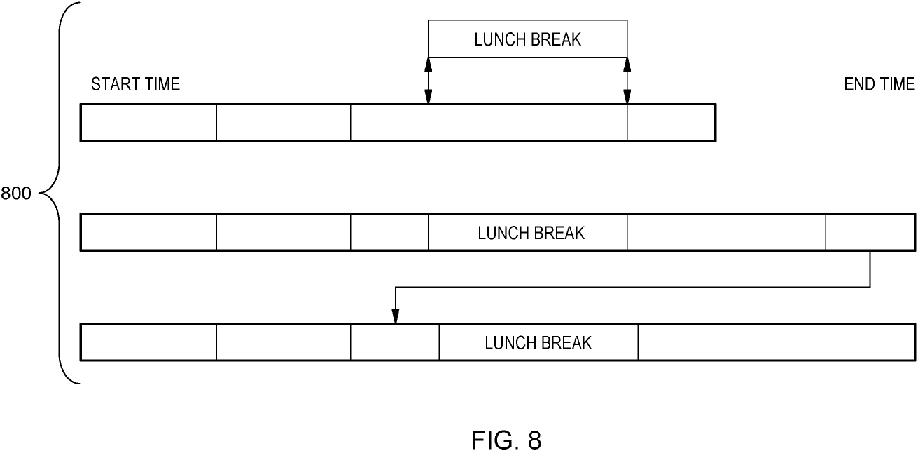

Diagram 600 of FIG. 6 and diagram 700 of FIG. 7 may be helpful in understanding the three methods set forth in the preceding paragraphs. According to some embodiments of the present invention: PROD(u, v) = pdf of u + v (convolution of PDFs). Diagram 800 of FIG. 8 shows the results of some of the optional post-processing described above.

A method for personalized radiology worklist sorting, the method includes the following operations (not necessarily in the following order): (i) receiving a set of radiologists’ daily exam reading data divided by sub-specialty, including but not limited to modality, body part, RVU, study difficulty estimation, study description, other patient and study information (if available); (ii) receiving exam reading orders, turnaround times, interruptions and break schedules associated with the daily exam reading data; (iii) receiving diagnostic accuracy information (pathology/biopsy results, peer review scores, follow-up results) associated with the daily exam reading data; (iv) training a sequence model to predict optimal worklist ordering for turnaround time and for diagnostic accuracy for each radiologist sub-specialty using the received data; (v) training a sequence model to predict the most preferred worklist ordering for each radiologist; (vi) receiving a new worklist to sort according to optimal turnaround time, accuracy, or preference; and (vii) sorting the received worklist using the machine learning algorithms and adjusting the results for personalized break and typical interruptions schedule.

### IV. Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above - similar cautions apply to the term “embodiment.”

And/or: inclusive or; for example, A, B “and/or” C means that at least one of A or B or C is true and applicable.

Including / include / includes: unless otherwise explicitly noted, means “including but not necessarily limited to.”

Module / Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Computer: any device with significant data processing and/or machine readable instruction reading capabilities including, but not limited to: desktop computers, mainframe computers, laptop computers, field-programmable gate array (FPGA) based devices, smart phones, personal digital assistants (PDAs), body-mounted or inserted computers, embedded device style computers, application-specific integrated circuit (ASIC) based devices.

What is claimed is:

1. A computer-implemented method (CIM) for use by a first medical profession who reviews patient image sets, respectively including at least one medical image, for medical purposes, the CIM comprising:
receiving a historical image review data set including information indicative of: (i) the manner in which the first medical professional has scheduled review of patient data image sets, and (ii) the manner in which a plurality of medical professionals have scheduled review of patient image sets;
determining, by machine logic, a set of at least one personalized scheduling rule(s) based on the historical image data review set;
receiving a first plurality of patient image sets respectively including one or medical image(s) of a respective patient; and
scheduling review of the first plurality of patient image sets based, at least in part, upon the personalized scheduling rules to obtain a first schedule for a first time period for the first medical professional to review the first plurality of patient image sets.

2. The CIM of claim 1 wherein the set of at least one personalized scheduling rule(s) includes a first rule that helps determine scheduling based on attribute(s) of the medical images of the first plurality of patient image sets.

3. The CIM of claim 1 wherein the attribute(s) of the medical images include image modality.

4. The CIM of claim 1 wherein the set of at least one personalized scheduling rule(s) includes a first rule that helps determine scheduling based on attribute(s) of patients imaged in the first plurality of patient image sets, with the first rule being based on patterns present in the historical image review data set.

5. The CIM of claim 1 wherein the scheduling of review of the first plurality of patient image sets includes only a determination of the time order of review of the patient image sets relative to each other and does not schedule times, dates or durations for the review of the respective patient image set.

6. The CIM of claim 1 further comprising:
receiving a set of baseline scheduling rules;
wherein the scheduling of the review of the first plurality of patient image sets is further based upon the baseline scheduling rules.

7. A computer-implemented method (CIM) for use by a first medical profession who reviews patient image sets, respectively including at least one medical image, for medical purposes, the CIM comprising:
receiving a historical image review data set including information indicative of the manner in which the first medical professional has scheduled review of patient data image sets;
determining, by machine logic, a set of at least one personalized scheduling rule(s) based on the historical image data review set;
receiving a first plurality of patient image sets respectively including one or medical image(s) of a respective patient; and
scheduling review of the first plurality of patient image sets based, at least in part, upon the personalized scheduling rules to obtain a first schedule for a first time period for the first medical professional to review the first plurality of patient image sets.

8. The CIM of claim 7 wherein the set of at least one personalized scheduling rule(s) includes a first rule that helps determine scheduling based on attribute(s) of the medical images of the first plurality of patient image sets, with the first rule being based on patterns present in the historical image review data set.

9. The CIM of claim 7 wherein the attribute(s) of the medical images include image modality.

10. The CIM of claim 7 wherein the set of at least one personalized scheduling rule(s) includes a first rule that helps determine scheduling based on attribute(s) of patients imaged in the first plurality of patient image sets.

11. The CIM of claim 7 wherein the scheduling of review of the first plurality of patient image sets includes only a determination of the time order of review of the patient image sets relative to each other and does not schedule times, dates or durations for the review of the respective patient image set.

12. The CIM of claim 7 further comprising:
receiving a set of baseline scheduling rules;
wherein the scheduling of the review of the first plurality of patient image sets is further based upon the baseline scheduling rules.

13. A computer-implemented method (CIM) for use by a first medical profession who reviews patient image sets, respectively including at least one medical image, for medical purposes, the CIM comprising:
receiving a historical image review data set including information indicative of the manner in which a plurality of medical professionals have scheduled review of patient image sets;
determining, by machine logic, a set of at least one personalized scheduling rule(s) based on the historical image data review set;
receiving a first plurality of patient image sets respectively including one or medical image(s) of a respective patient; and
scheduling review of the first plurality of patient image sets based, at least in part, upon the personalized scheduling rules to obtain a first schedule for a first time period for the first medical professional to review the first plurality of patient image sets.

14. The CIM of claim 13 wherein the set of at least one personalized scheduling rule(s) includes a first rule that helps determine scheduling based on attribute(s) of the medical images of the first plurality of patient image sets.

15. The CIM of claim 14 wherein the attribute(s) of the medical images include image modality.

16. The CIM of claim 13 wherein the set of at least one personalized scheduling rule(s) includes a first rule that helps determine scheduling based on attribute(s) of patients imaged in the first plurality of patient image sets, with the first rule being based on patterns present in the historical image review data set.

17. The CIM of claim 13 wherein the scheduling of review of the first plurality of patient image sets includes only a determination of the time order of review of the patient image sets relative to each other and does not schedule times, dates or durations for the review of the respective patient image set.

18. The CIM of claim 13 further comprising:
receiving a set of baseline scheduling rules;
wherein the scheduling of the review of the first plurality of patient image sets is further based upon the baseline scheduling rules.
